(19) United States
(12) Patent Application Publication
(10) Pub. No.: US 2008/0058883 A1
(43) Pub. Date: Mar. 6, 2008

(54) METHOD AND SYSTEM TO CONTROL EARLY BATTERY END OF LIFE EVENTS ON MULTI-TRANSCEIVER SYSTEMS

(75) Inventors: ROBERTO GAUTIER, DAVIE, FL (US); F. W. CHERRY, COOPER CITY, FL (US); DAVID R. HEESCHEN, CORAL SPRINGS, FL (US); ASHLEY JAMES J. LAWRIE, OAKDEN (AU); JOSE F. RODRIGUEZ, HIALEAH, FL (US); STEFAN PETKOV P. STEFANOV, SHEIDOW PARK (AU); JASON T. YOUNG, PALM CITY, FL (US)

Correspondence Address: AKERMAN SENTERFITT, P.O. BOX 3188, WEST PALM BEACH, FL, 33402-3188, US

(73) Assignee: MOTOROLA, INC., SCHAUMBURG, IL (US)

(21) Appl. No.: 11/468,071

(22) Filed: Aug. 29, 2006

(51) Int. Cl. A61N 1/00

(52) U.S. Cl. 607/29

(57) ABSTRACT

A mobile device (102) and method (500) for handling early end of life (EOL) battery indications is provided. The method can include receiving (502) an indication for an end of life (EOL) event, determining (504) an operating mode (704/706) in view of the EOL event (708), determining (506) whether the mobile device is in multiple transmit communication (710), evaluating (508) a battery level of the mobile device, and performing (510) an action (714) in response to the EOL event for mitigating an early shutdown of the mobile device. An action can include masking a low battery alert corresponding to the EOL event, limiting at least one service associated the operating mode, or shutting down the mobile device.

### FIELD OF THE INVENTION

This invention relates generally to communication systems, and more particularly to power management.

### BACKGROUND OF THE INVENTION

The mobile device industry is constantly challenged in the market place for high quality, low-cost products having strong battery life characteristics. Moreover, demand for mobile devices which allow users to stay continually connected has dramatically risen. Service providers and manufacturers are offering more services over more networks for keeping users connected. Mobile devices, such as a radio or cell phone, can include a transceiver for providing receive and transmit communication operations. The mobile devices may be capable of supporting multiple processing cores and providing multiple simultaneous communications. In order to achieve “seamless mobility”, and allowing users to stay continually connected, a mobile device can employ multiple transceivers that operate on different networks. The networks may each operate in an asynchronous manner such that the mobile device interacts with each network in an independent manner. That is, each transceiver may operate independently of the other transceivers on the mobile device, draining power from the battery as needed. Continued operation of multiple transceivers for providing simultaneous communications can drain the battery life of the mobile device. Consequently, the mobile device may perform an early shutdown if the battery voltage decreases below an end of live level, even though additional battery capacity remains. The early shutdown terminates any services associated with the transceivers. A need therefore exists for mitigating early shutdown when additional battery capacity remains.

### SUMMARY OF THE INVENTION

One embodiment of the invention is directed to a method to handle early end of life (EOL) battery indications on a mobile device. The method can include receiving an indication for an end of life (EOL) event, determining an operating mode in view of the EOL event, determining whether the mobile device is in multiple transmit communication, evaluating a battery level of the mobile device, and performing an action in response to the EOL event for mitigating an early shutdown of the mobile device based on the operating mode, the battery level, and the multiple transmit communication for allowing continued current drain on the battery and prolonging use of the mobile device. An EOL event can be evaluated to determine it the EOL event was due to EOL multi-transceiver activity. If so, an unloaded battery voltage can be measured to determine if additional battery capacity is available. If so, an operating mode of a transceiver can be changed to reduce a loading on the battery without affecting a service of another transceiver. An operating mode can be an idle mode, an active mode, or an airplane mode. Performing an action can include masking a low battery alert corresponding to the EOL event, limiting a service associated an operating mode of a transceiver, or shutting down the mobile device. For example, a service associated with the EOL event can be determined, and the service can be limited by changing at least one operating mode during the multiple transmit communication.

Embodiments of the invention are also directed to a method for handling low battery conditions on a mobile device. The method can include monitoring the mobile device for an end of life (EOL) event, determining an operating mode of at least one transceiver associated with the EOL event, determining whether the EOL event is due to a multi-transceiver configuration, evaluating a battery level of the mobile device, and performing an action in response to the EOL event for mitigating an early shutdown of the mobile device based on the operating mode, the multi-transceiver configuration, and the battery level. If the device is operating in a multi-transceiver configuration, a battery capacity can be measured, the battery capacity can be compared to at least one threshold, and an operating mode of the mobile device can be changed if the battery capacity exceeds at least one threshold in response to the EOL event.

Embodiments of the invention are also directed to a mobile device to control early battery end of life events. The mobile device can include a battery for providing power to the mobile device, at least one transceiver cooperatively coupled to the battery for providing communications, and a processor coupled to the at least one transceiver and the battery. The processor can monitor an end of life (EOL) event due to multiple transmit activity of the at least one transceiver, determine an operating mode of the at least one transceiver associated with the EOL event, and mask the EOL event in view of the operating mode. A transceiver can generate an interrupt in response to a current drain on the battery which flags the EOL event. The mobile device can include a battery indicator for measuring a voltage of the battery. The processor can compare the battery voltage to at least one threshold, and control an operating mode of the mobile device if the battery voltage falls below at least one threshold.

### BRIEF DESCRIPTION OF THE DRAWINGS

FIG. 1 is a diagram of a mobile communication system within a mobile communication environment;

FIG. 2 is a schematic of a mobile device in accordance with the embodiments of the invention;

FIG. 3 is a schematic of a simplified battery model in accordance with the embodiments of the invention;

FIG. 4 is a plot of a battery voltage with respect to multiple transceiver operation in accordance with the embodiments of the invention;

FIG. 5 is a method for handling early end of life (EOL) battery indications on a mobile device in accordance with the embodiments of the invention;

FIG. 6 is a plot of transceiver duty cycles in accordance with the embodiments of the invention;

FIG. 7 is a truth table decision matrix in accordance with the embodiments of the invention;

FIG. 8 is a comparison of end of life thresholds and a decision threshold in accordance with the embodiments of the invention;

FIG. 9 is flowchart for handling low battery conditions on a mobile device in accordance with the embodiments of the invention;

FIG. 10 is a hardware detector circuit for determining multiple transmit operations in accordance with the embodiments of the invention;

FIG. 11 is an algorithm for dynamic end of life thresholds in accordance with the embodiments of the invention;

### DETAILED DESCRIPTION OF THE DRAWINGS

While the specification concludes with claims defining the features of the embodiments of the invention that are regarded as novel, it is believed that the method, system, and other embodiments will be better understood from a consideration of the following description in conjunction with the drawing figures, in which like reference numerals are carried forward.

As required, detailed embodiments of the present method and system are disclosed herein. However, it is to be understood that the disclosed embodiments are merely exemplary, which can be embodied in various forms. Therefore, specific structural and functional details disclosed herein are not to be interpreted as limiting, but merely as a basis for the claims and as a representative basis for teaching one skilled in the art to variously employ the embodiments of the present invention in virtually any appropriately detailed structure. Further, the terms and phrases used herein are not intended to be limiting but rather to provide an understandable description of the embodiment herein.

The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “suppressing” can be defined as reducing or removing, either partially or completely. The term “processor” can be defined as any number of suitable processors, controllers, units, or the like that carry out a pre-programmed or programmed set of instructions. The term “idle mode” can be defined as not currently in a call. The term “active mode” can be defined as currently in a call or providing a service. The term “airplane mode” can be defined as providing computational functions but not providing transmit and receive operations. The term “EOL” can be defined as nearing an end of battery life. The term “battery capacity” can be defined as the voltage across a battery. The term “loaded” can be defined as placing a resistive or current load on a battery. The term “unloaded” can be defined as an open voltage across the leads of a battery. The term “service” can be defined as providing data for a communication process. The term “transceiver” can be defined as a hardware or software controlled component capable of performing receive and transmit communication functions. The term “multi-transceiver” can be defined as a component having multiple transceivers capable of performing independent receive and transmit functions simultaneously. The term “shutdown” can be defined as ending an operation or terminating a process of a processor. The term “masking” can be defined as suppressing a visual presentation of an early battery indication, bypassing a shutdown of a processor, changing an operating mode to mitigate excessive loading of a battery, or limiting a service to prevent an early end of life. The term “early” can be defined as occurring prematurely. The term “early end of life” can be defined as shutting down a processor even though a battery charge still remains. The term “interrupt” can be defined as temporarily flagging a service request. The term “multiple transmit” operation can be defined as transmitting two or more multiple communications. Similarly, the term “multiple transmit” can include two or more transmitters actively transmitting at the same time. The term “dual” can be defined as two or more.

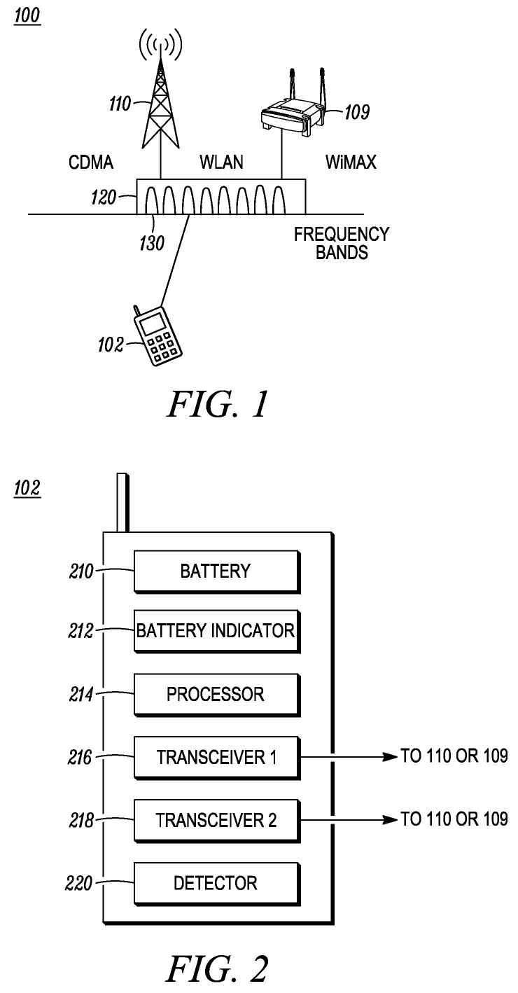

Referring to FIG. 1, a mobile communication system 100 for providing mobile communication is shown. The mobile communication system 100 can include one or more subscribers, such as mobile device 102. A mobile device can be a radio, a cell phone, a personal digital assistant, a mobile communication device, a public safety radio, a portable media player, an emergency communication device, or any other suitable communication device. In one arrangement, more than one mobile device can operate within the mobile communication environment for providing group call or dispatch communication. The mobile device 102 may include one or more transceivers for providing multiple simultaneous communications. For example, a first transceiver can establish and handle a phone call, and a second transceiver can handle email messaging updates. Notably, multiple transceivers on a single mobile device 102 can handle various processing tasks, and are not limited to those disclosed herein.

The mobile communication system 100 can provide wireless connectivity over a radio frequency (RF) communication network such as a base station 110, also known as a tower. The base station 110 may also be a base receiver, a central office, a network server, or any other suitable communication device or system for communicating with the one or more mobile devices. The mobile device 102 can communicate with one or more cellular towers 110 using a standard communication protocol such as Time Division Multiple Access (TDMA), Global Systems Mobile (GSM), integrated Dispatch Enhanced Network (iDEN), Code Division Multiple Access (CDMA), Orthogonal Frequency Division Multiplexing (OFDM) or any other suitable modulation protocol. The base station 110 can be part of a cellular infrastructure or a radio infrastructure containing standard telecommunication equipment as is known in the art.

In another arrangement, the mobile device 102 may also communicate over a wireless local area network (WLAN). For example the mobile device 102 may communicate with a router 109, or an access point, for providing packet data communication. In a typical WLAN implementation, the physical layer can use a variety of technologies such as 802.11b or 802.11g Wireless Local Area Network (WLAN) technologies. As an example, the physical layer may use infrared, frequency hopping spread spectrum in the 2.4 GHz Band, or direct sequence spread spectrum in the 2.4 GHz Band, or any other suitable communication technology.

In particular, the base station 110, or the router 109, can support one or more frequency bands 130 to the plurality of mobile devices 102 and 104. A frequency band can include CDMA, OFDM, WLAN, or WiMAX but is not herein limited to these. Frequency bands can include UHF and VHF for short range communication. In general, the base station 110 or the router 109 will be responsible for allocating one or more frequency channels 130 to the mobile device 102. Once assigned one or more frequency channel 130, the mobile device 102 can communicate over the network using the one or more assigned frequency channel. Notably, depending on the form of communication, various frequency channels may be available. That is, the mobile device 102 may be capable of operating over multiple frequency channels. The mobile device 102 can also receive communication over the assigned frequency channel. A mobile device 102 have multiple transceivers can communicate simultaneously over one or more frequency channels 120.

The mobile device 102 can receive communication signals from either the base station 110 or the router 109. Other telecommunication equipment can be used for providing communication and embodiments of the invention are not limited to only those components shown. As one example, the mobile device 102 may receive a UHF radio signal having a carrier frequency of 600 MHz, a GSM communication signal having a carrier frequency of 900 MHz, or an IEEE-802.11x WLAN signal having a carrier frequency of 2.4 GHz. Notably, the mobile device 102 can include multiple transceivers for providing multiple simultaneous communications. For example, the mobile device 102 can handle a phone call over a GSM transceiver connection, and a network mobility update for location awareness or presence on the WLAN transceiver connection.

Referring to FIG. 2, a block diagram of the mobile device 102 is shown. The mobile device 102 can include a battery 210 for providing power to the mobile device, a battery indicator 212 coupled to the battery for identifying a voltage of the battery 210, and a processor 214 powered by the battery for controlling mobile device operation. In particular, the processor 214 can identify end of life events and make a determination to mask an end of life event depending on an operating mode of the mobile device 102. An end of life (EOL) event occurs when excessive loading on the battery 210 causes a battery voltage to decrease below a specific threshold. For example, multiple processes by multiple transceivers can cause excess current drain on the battery 210 which can trigger an EOL event. Upon receiving an EOL event, the processor 214 may shutdown.

The mobile device 102 can include a first transceiver 216 for providing a first communication and a second transceiver 216 for providing a second communication as discussed in FIG. 1. Notably, the mobile device 102 may include more transceivers than those shown, or the transceivers may be integrated on a single core. For example, an integrated circuit (IC) may include a plurality of receivers, transmitters, amplifiers, analog-to-digital converters, and filters for providing various communication functions. The IC may be a single composite element that provides multiple transceivers capable for supporting simultaneous communication processes. The mobile device can further include a detector 220 cooperatively coupled to the first transceiver 216, the second transceiver 218, and the processor 214 for determining if the mobile device is operating in a multi-transceiver mode; that is, if the first transceiver 216 and the second transceiver 218 are operating simultaneously.

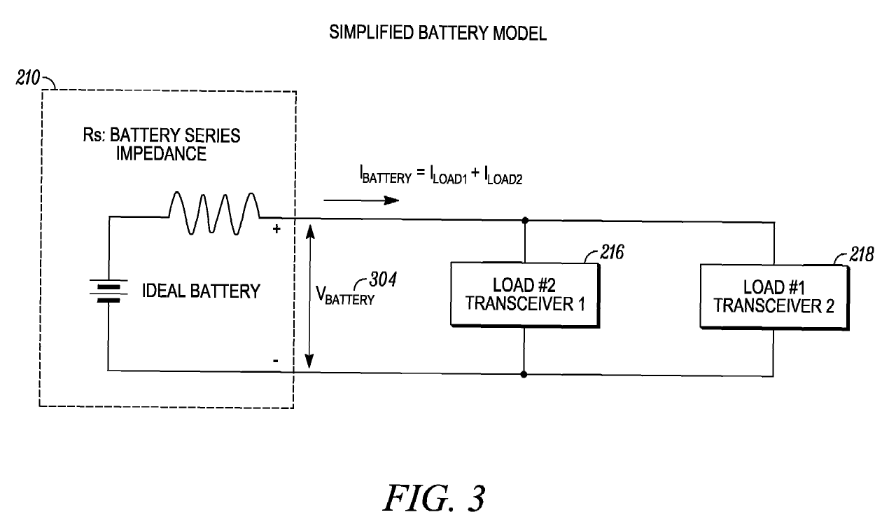

Briefly referring to FIG. 3, a simplified battery model 300 showing multiple loading on the battery 210 is shown. The simplified battery model 300 illustrates loading effects when the first transceiver 216 and the second transceiver 218 (See FIG. 2) are transmitting simultaneously. Briefly described, each transceiver imparts a load effect on the battery 210 that drains the battery current. Consequently, the battery voltage 304 can decrease substantially when two loads are connected in parallel to the battery 210. For example, if the loads are considered resistive loads each having a resistance R, the overall resistance seen by the battery 210 can decrease to R/2. Accordingly, the multiple loading draws more current from the battery which in turns lowers the battery voltage.

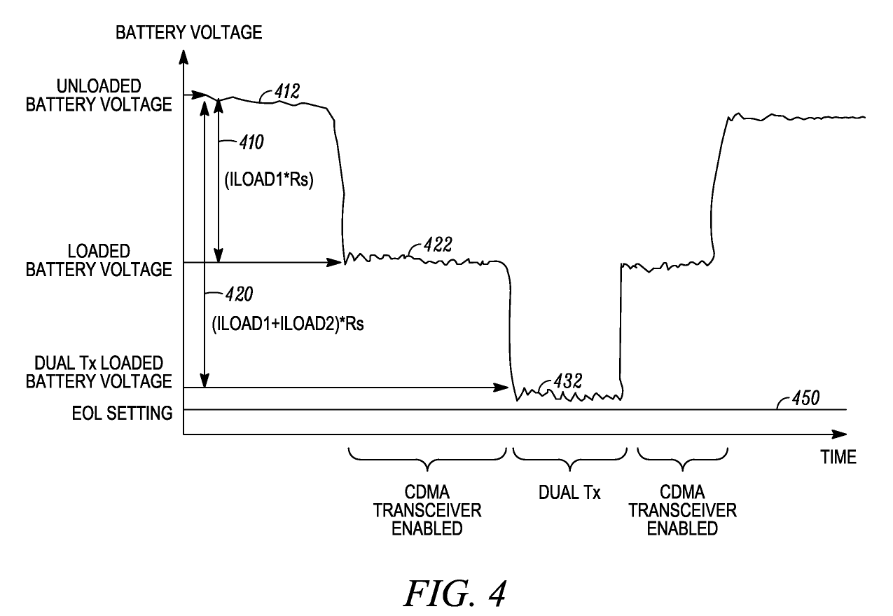

Referring to FIG. 4, a plot 400 of the battery voltage with respect to multiple transceiver operation in time is shown. In particular, the plot 400 illustrates the change in voltage level due to multiple transceiver loading on the battery 210 (See FIG. 3). In particular, an unloaded battery 412 has an open voltage corresponding to the voltage across the terminals of the battery 210 (See FIG. 3). The battery 210 is considered loaded when a load is placed across the battery terminals. When the first transceiver 216 is currently active, it will load the battery 210 and lower the battery voltage to 422. For example, the first transceiver 216 may be providing iDEN communications. If the second transceiver 218 becomes active while the first transceiver 216 is operating, the net effect will be an overall decrease in voltage to 432. For example, the second transceiver 218 may be providing CDMA communications. If the battery voltage decreases below a certain threshold corresponding to an EOL setting 450, an EOL event can be triggered. For example, referring back to FIG. 2, the processor 214 can issue an EOL event when the voltage level falls below the EOL setting 450 and shutdown either or both of the transceivers, 216 and 218. In operation, when the loaded battery voltage falls below the EOL setting 450, regulator circuits associated with the transceivers may stop working and the circuits will operate out of specification. At this point, an EOL detector in the processor 214 may activate and begin shutdown of the mobile device 102.

It should be noted that multiple loading can give a false impression that the battery voltage is below battery capacity. That is, there can be instances where battery capacity can be perceived as low as a result of the battery voltage decreasing due to temporary multiple loading of the transceivers. The multiple transmit operation may temporarily result in a voltage level 432 that may be lower than the EOL setting 450. Notably, the battery can still provide operations, but just not for multiple transceiver loads which indicates a low battery level. The battery may still be able to operate with a reduced load which will occur when one of the transceivers terminates communication. However, the mobile device will not be able to continue operating when the EOL event triggers a shutdown. Accordingly, one embodiment of the invention is directed to masking an EOL event when a multiple transmit communication is detected thereby allowing the mobile to continue operation when multiple transmit communication subsides.

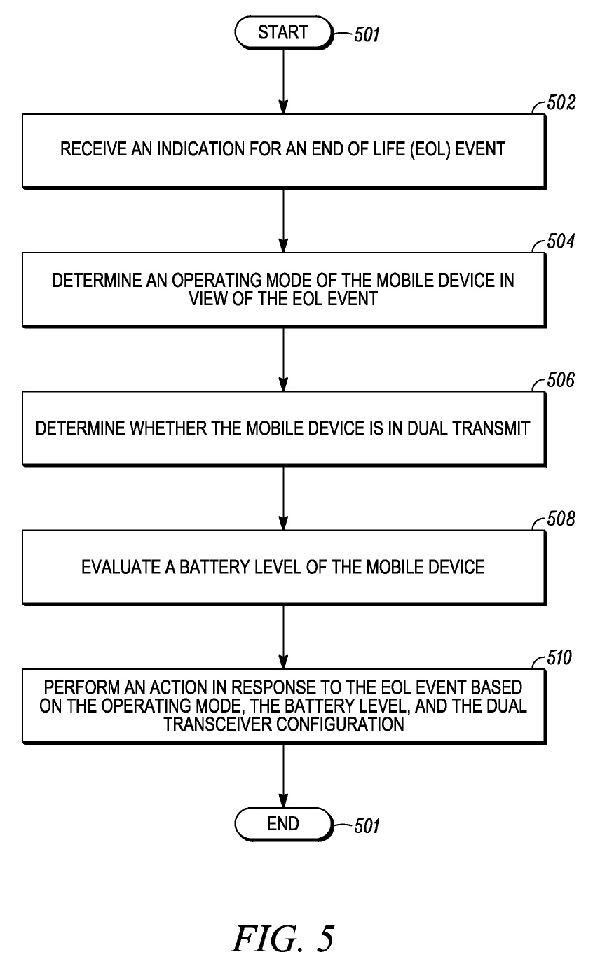

Referring to FIG. 5, a method 500 for handling early end of life (EOL) battery indications on a mobile device is shown. The method 500 can be practiced with more or less than the number of steps shown. To describe the method 500, reference will be made to FIGS. 2, 4, 6, and 7 although it is understood that the method 500 can be implemented in any other suitable device or system using other suitable components. Moreover, the method 500 is not limited to the order in which the steps are listed in the method 500. In addition, the method 500 can contain a greater or a fewer number of steps than those shown in FIG. 5.

At step 501, the method 500 can start. The method can start in a state wherein a mobile device receives an indication of a low battery mode. The low battery mode may be a result of low battery capacity, or multiple transceiver loading on the battery. At step 502, an indication can be received for an end of life (EOL) event. The EOL event indicates that the battery voltage has fallen below a threshold level. A hardware EOL circuit resident on a transceiver may automatically generate the EOL event when the battery voltage to the transceiver falls below an internal EOL threshold. Recall, however, that the battery capacity may be sufficient to continue operation if the battery voltage drop is due to multiple transceiver loading. For example, referring back to FIGS. 2 and 4, the battery indicator 212 can report a battery voltage to the processor 214. The battery indicator can measure a battery capacity by averaging multiple software readings of a voltage of the battery.

At step 504, an operating mode can be determined in view of the EOL event. An operating mode can be an idle mode, an active mode, or an airplane mode but is not herein limited to these. Referring to FIG. 2, the processor 214 can determine the operating mode of the mobile device 102. An operating mode can identify a state of the first transceiver 216 and a state of the second transceiver 218. In particular, each transceiver can be in an operating mode. For example, the first transceiver 216 can be in active mode, such as in a call. The second transceiver 218 can also be in an active mode such as performing network mobility updates, or in an idle mode.

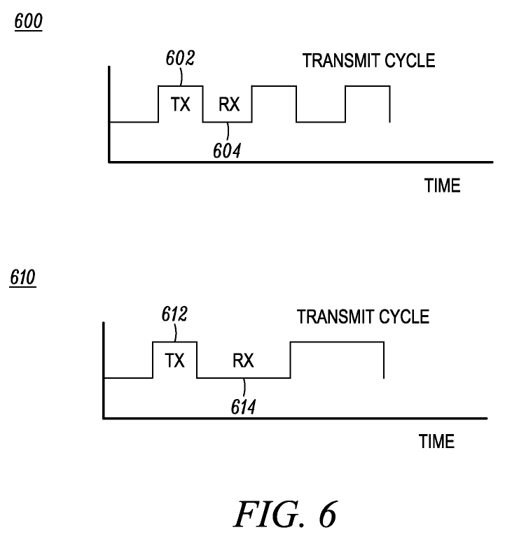

At step 506, a determination can be made as to whether the mobile device is in multiple transmit communication. Briefly, the operating modes determined in step 502 identify a state of the first 216 and second 218 transceivers, but do not generally indicate whether the transceivers are operating simultaneously. For example, referring to FIG. 6, a first duty cycle 600 for the first transceiver 216 and a second duty cycle 620 for the second transceiver 218 is shown. A duty cycle identifies the transmit and receive periods for a transceiver. For example, first transceiver 216 may transmit at a first time 602 and receive at a second time 604. Second transceiver 218 may transmit at a first time 612 and receive at a second time 614. The transmit cycles may coincide at various times depending on the communication processes implemented by the transceivers. For example, first transmit time 602 of the first transceiver 216 may coincide with the second transmit time 612 of the second transceiver 218. When both transmit times coincide, this can cause excessive concurrent loading on the battery as described in FIG. 4. For example, when first transmit time 602 and second transmit time 604 coincide, the battery voltage can decrease to 432 and approach the EOL setting 450.

Returning back to the discussion of method 500 of FIG. 5, at step 508, a battery level of the mobile device can be evaluated. For example, referring back to FIG. 2, the battery indicator 212 can measure the voltage of the battery 210. At step 510, an action can be performed in response to the EOL event for mitigating an early shutdown of the mobile device based on the operating mode, the battery level, and the multiple transmit communication. The action can allow continued current drain on the battery and prolong use of the mobile device. An action can include masking a low battery alert corresponding to the EOL event, limiting at least one service associated the operating mode, or shutting down the mobile device. In operation, referring back to FIG. 2, the processor 214 can monitor an end of life (EOL) event, determine an operating mode of a transceiver associated with the EOL event, and mask the EOL event based on the operating mode.

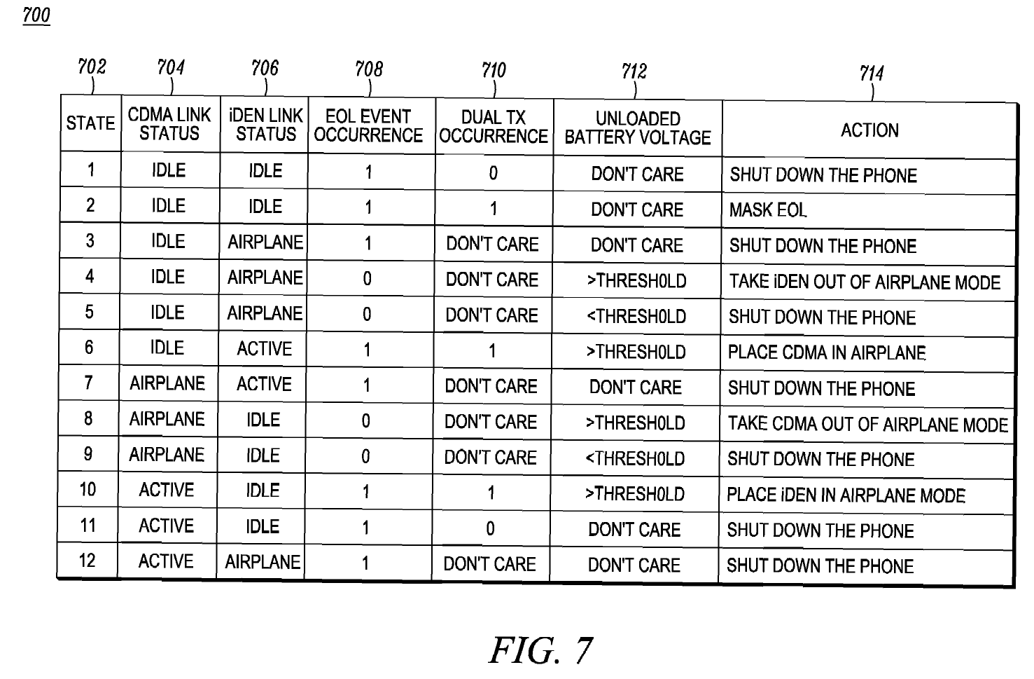

Referring to FIG. 7, an exemplary truth table decision matrix 700 is shown that outlines actions performed by the processor in response to receiving an early EOL battery indication. Briefly, once an EOL event is triggered, the processor 214 (See FIG. 2), determines the states (e.g. operating modes) of the transceivers that caused the EOL event. Once the states are identified, the processor 214 determines the course of action based on the truth table 700 to allow the user to continue operation with a present service, limit a current service, or shutdown the mobile device. For example, the processor 214 can identify operating modes, and change an operating mode to reduce a loading on the battery. In particular, the truth table 700 identifies the course of actions taken by the processor 214.

Notably, the truth table decision matrix 700 is merely an example set of actions, and a brief description of the truth table decision matrix is provided. The value “1” in the table represents a positive occurrence of an event or condition, and the value “0” represents a negative occurrence of the event or condition. The value “1” means TRUE (e.g. the event did occur), and the value “0” means FALSE (e.g. the event did not occur). The state 706 is simply an enumeration of the actions. The CDMA link status 704 is a first communication type for the first transceiver 216 (See FIG. 2). The iDEN link status 706 is a second communication type for the second transceiver 218 (See FIG. 2). The link status 704 and 706 list communication protocols of the transceivers by name for reference. The first transceiver 216 is not limited to only CDMA communications, and the second transceiver 218 is not limited to only iDEN communications. The link status 704 and 706 are merely provided as examples for showing the multiple transceiver nature of the mobile device 102.

The EOL event 708 occurs when excessive loading on the battery causes high current drain and lowers the overall battery voltage. The Multiple Tx Occurrence 710 identifies whether transceivers 216 and 218 are operating simultaneously. For example, referring back to FIG. 2, the detector 220 determines whether the first transceiver 216 and the transceiver 218 are transmitting simultaneously as seen by the duty cycles 600 and 610 in FIG. 6. The unloaded battery voltage 712 identifies whether the battery voltage is below a threshold or above a threshold. Depending on certain entries (e.g. 0 or 1) of the EOL event 708 and Multiple Tx Occurrence 710, the unloaded battery voltage 712 may be a “don't care” situation. For example, in state 1 (702), if both transceivers are in idle mode, then an only option when an EOL event is received is to shut down the phone. The unloaded battery voltage does not affect the decision to shut down. That is, there is no additional battery capacity as the transceivers are already in a low battery operating state and not transmiting simultaneously. In state 2 (702), if both transceivers are in idle mode and simultaneously transmitting (i.e. Multiple Tx=1 [710]), then the EOL event can be masked to mitigate early shutdown. In this case, the EOL event is due to concurrent transmit operations by the first and second transceiver. The multiple transmit gives a false impression that the battery voltage is below capacity. However, the mobile device is capable of providing communication as long as the transceivers are not transmitting simultaneously. By masking the EOL event, the mobile device can be preempted from entering early shutdown. Accordingly, the mobile device can resume operation due to the masking of the EOL event. As another example, at state 6, the first transceiver 216 providing the CDMA link may be idle (704), and the second transceiver 218 providing the iDEN link may be active (706). If an EOL event occurs (708) and a Multiple Tx Occurs (710), then a determination can be made as to whether battery capacity is available. For example, the unloaded battery voltage 712 can be compared to a threshold to determine if an operating mode of a transceiver can be changed to mitigate early shutdown and prolong use of the mobile device. If the battery voltage is greater than the threshold (712), the mobile device can place the first transceiver providing the CDMA link in airplane mode?. That is, the first transmitter 216, though operating in idle mode, can be placed in a lower operating state such as airplane mode to reduce loading on the battery. In idle mode, the transceiver may still receive and transmit communications. However, in airplane mode, the receivers and transmitters are disabled thereby reducing loading on the battery and preventing simultaneous transmit or receive operations causing EOL events.

Understandably, each of the states 702 and the associated actions 714 of the truth table decision matrix 700 can be considered with respect to the transceiver link status (704 and 706), the EOL event 708, the Multiple Tx Occurrence 710, and the unloaded battery voltage.

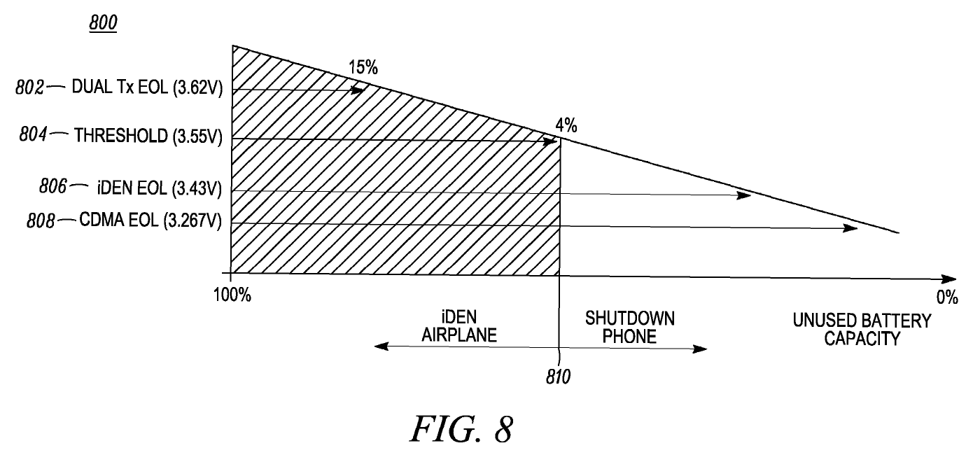

Briefly referring to FIG. 8, a comparison of EOL thresholds is illustrated for understanding entries in the truth table 700 of FIG. 7. In particular, each transceiver may have a corresponding EOL threshold at which point an EOL event is triggered. For example, during multiple transmit operation when both the first transceiver 216 and the second transceiver 218 (See FIG. 2) are communicating simultaneously, an EOL event (502) will be triggered if the unloaded battery voltage falls below 802. This would result in an early EOL shutdown while still having battery capacity left for single mode operation (812). When only the second transceiver 218 is operating with iDEN link status 706 (See FIG. 7), an EOL event (502) will be triggered if the unloaded battery voltage falls below 806. When only the first transceiver 216 is operating with CDMA link status 704 (See FIG. 7), an EOL event (502) will be triggered if the unloaded battery voltage falls below 808. Notably, a threshold 804 is introduced to identify when a course of action can be undertaken to mitigate early shutdown due to an EOL event. For example, the threshold 804 is positioned greater than the single transceiver EOL event thresholds (806 and 808) but lower than the Multiple Tx EOL threshold 802. In the case where both transceivers are operating simultaneously, one of the operating modes of one of the transceivers can be adjusted to reduce the loading on the battery without changing the service offered by the other transceiver. For example, if the first transceiver 216 is in a call on the CDMA link status (706), the second transceiver 218 is receiving mobility updates, and both transceivers are transmitting simultaneously, an operating mode of the second transceiver can be adjusted to reduce loading on the battery. As an example, the second transceiver 218 can be placed in airplane mode to reduce loading without affecting the CDMA call on the first transceiver 216. That is, if the battery voltage is less than the threshold 804, and Multiple Tx Occurrence is true (i.e. set to 1), then the second transceiver can be placed in airplane mode, corresponding to the left side of line 810. If the battery voltage is lower than the threshold 804, and Multiple Tx Occurrence is true (i.e. set to 1), then the battery capacity cannot be prolonged and a shutdown occurs, corresponding to the right side of line 810.

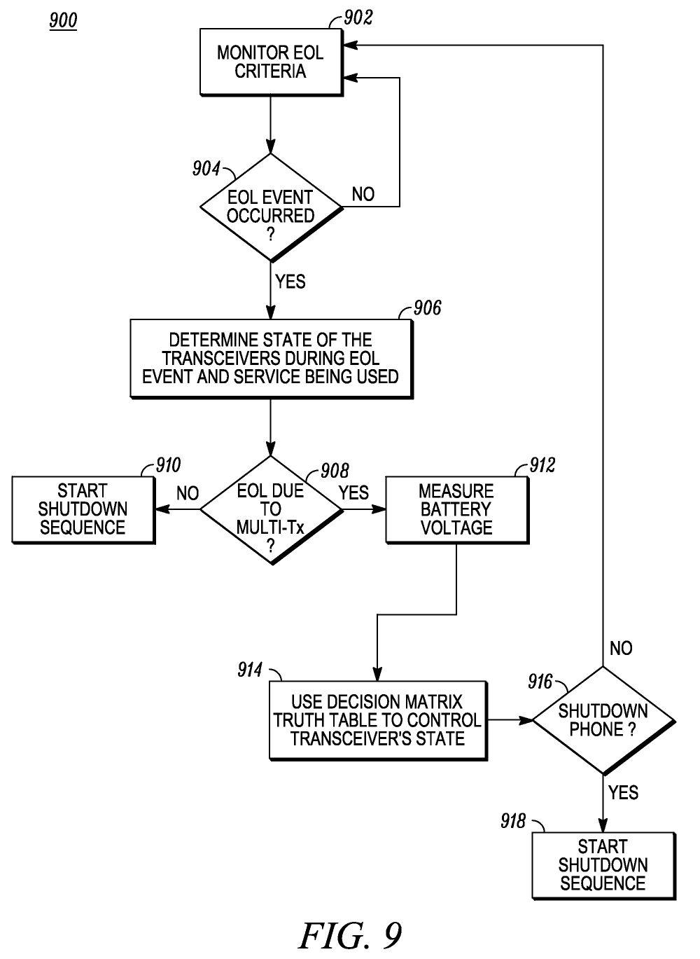

Referring to FIG. 9, a flow chart 900 for handling low battery conditions on a mobile device is shown. In particular, the flowchart 900 summarizes a state logic for the truth table decision matrix 700 of FIG. 7. The flow chart 900 can be practiced with more or less than the number of steps shown. To describe the flow chart 900, reference will be made to FIGS. 2, 7, and 10 although it is understood that the flow chart 900 can be implemented in any other suitable device or system using other suitable components. Moreover, the flow chart 900 is not limited to the order in which the steps are listed in the flow chart 900. In addition, the flow chart 900 can contain a greater or a fewer number of steps than those shown in FIG. 9.

At step 901, EOL criteria can be monitored. For example, referring back to FIG. 2, the processor 214 can monitor EOL events. At step 902, a determination can be made as to whether an EOL even occurred. If an EOL event does not occur, the flow can return to step 901. If an EOL event does occur, the flow can proceed to step 906. At 906, a state of the transceivers can be determined during the EOL event and a service during the EOL event can be determined. For example, an operating mode of the transceivers can be determined for identifying the state. Referring back to FIG. 7, the transceivers may be in an idle mode, an active mode, or an airplane mode. Each of the transceivers may also be providing a service that may be dependent on the mode. For example, the first transceiver 216 providing the CDMA link (704) may be in an active mode and supporting a phone call as the service. The second transceiver 218 providing the iDEN link (706) may be in an idle mode and providing update services. At step 908, a determination can be made as to whether the EOL event was due to multi-transceiver operation; that is, is the first transceiver 216 and the second transceiver 218 transmitting or receiving simultaneously. At step 910, the mobile device can undergo a shut down sequence if the EOL is not due to multi-transmit mode. In one arrangement, referring back to FIG. 7, the Multiple Tx Occurrence entry 710 identifies whether the transceivers are operating simultaneously.

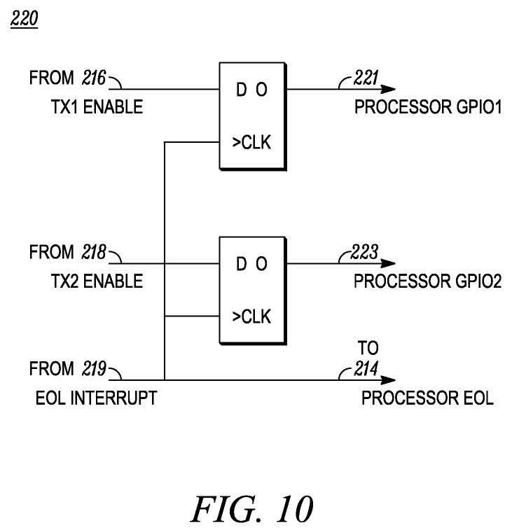

Briefly referring to FIG. 10, a detector circuit 220 is shown as on example for determining whether the first and second transmitters are operating simultaneously. Method steps 912-918 of FIG. 9 will be included in the description of FIG. 10. The detector circuit 220 can be software defined or hardware defined. As illustrated, the detector circuit 220 is a hardware circuit comprising two D flip-flops but is not limited to the arrangement shown. The detector circuit 220 provides a latching mechanism to latch a state of the transmitter control signals generated by the first and second transceivers. The hardware circuit 220 can receive a first input from a control line of the first transceiver 216, a second input from a control line of the second transceiver 218, and an EOL hardware detector interrupt 219. The latched control lines 221 and 223 can be read via general purpose input/output (GPIO) signals on the processor 214 to determining if an EOL event was caused by the transceivers.

If the transceivers are not operating simultaneously (908), the mobile device 102 can be shut down (910). If the transceivers are operating simultaneously (e.g. Multiple Tx Occurrence=1 [710]), the EOL event may be masked. Foremost, a determination can be made as to the battery voltage. Accordingly, referring back to FIG. 9, at step 912 a battery level can be measured. At step 914, the truth table 700 of FIG. 7 can be referenced as a decision matrix to determine a course of action. A course of action can include masking a low battery alert corresponding to the EOL event, limiting at least one service associated the operating mode, or shutting down the mobile device. At step 916, a determination can be made to shut down the phone based on the actions outlined by the truth table decision matrix 700 of FIG. 7. In particular, a shutdown generally occurs when one of the transceivers cannot be placed in a mode that reduces the load on the battery. A masking event or a changing of an operating mode generally occurs when a course of action results in a transceiver operation that reduces a loading on the battery. For example, the mobile device 102 may indicate 1 bar of battery life on a display of the mobile device. A user of the mobile device may be on an active call using a first transceiver of the mobile device. If a second transceiver needs to perform a mobility update, the extra loading on the battery may decrease the voltage below an EOL threshold and cause the mobile device to shut down prematurely. The method 500 of FIG. 5 in conjunction with the truth table 700 of FIG. 7, determines a course of action that prevents the second transceiver from transmitting simultaneously with the first transmitter thereby mitigating premature shutdown. For example, the second transmitter is temporarily placed in airplane mode, until it can be taken out of airplane mode to avoid transmit contention with the first transmitter. In this case, the EOL event can be masked by preempting a simultaneous transmission. If however, the mobile device cannot mitigate an early EOL battery indication, a shutdown will resume. At step 918, the mobile device will start a shutdown sequence if an EOL event cannot be masked, a service cannot be limited, or an operating mode can not be changed to further reduce loading on the battery. If an early shutdown can be mitigated by any of the aforementioned approaches, the flow can resume to step 902 wherein the mobile device continues to monitor for EOL events.

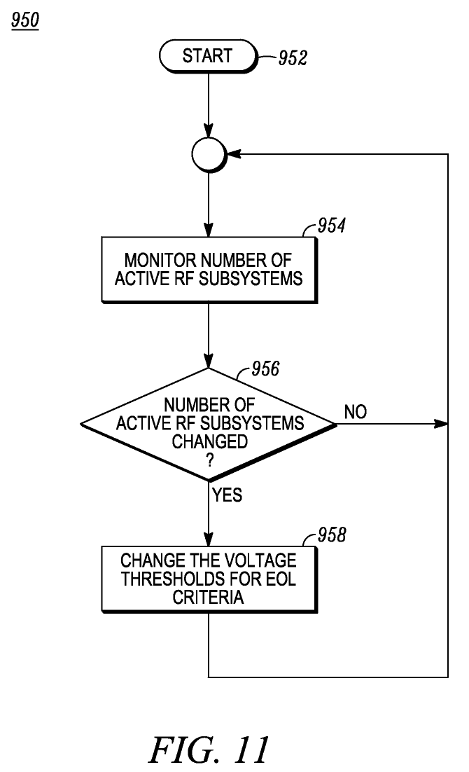

Referring to FIG. 11, a flexible threshold algorithm 950 is shown. The algorithm 950 provides a programmable shutdown voltage as an extension mechanism to a fixed threshold. That is, referring back to FIG. 8, the fixed threshold 804 can be programmable based on EOL thresholds, such as 802, 806, and 808. A programmable shutdown voltage allows the mobile device to dynamically set the shutdown at a lower voltage if a single RF subsection is active, or higher, if two or more RF modems are active. The programmable shutdown voltage depends on the requirements of the RF transceiver and base band processor.

The algorithm 950 can be used to monitor the EOL criteria as presented in step 902 of FIG. 9. In particular, the algorithm 950 can run asynchronously and simultaneously with a monitoring of EOL events. That is, the process step 902 to monitor EOL criteria can reference the dynamically varying voltage shutdown thresholds set by the algorithm 950. Additional logic (not shown) can be added to set the voltage thresholds for EOL events depending on which combination of RF transceivers are simultaneously active and their respective minimum operating voltage requirements. For example, a WiMAX/iDEN/CDMA threshold can correspond to a highest shutdown voltage of the either WiMAX, iDEN, or CDMA alone which may not necessary be the same if another combinations of modems was active, for example iDEN/CDMA/Bluetooth.

For example, at step 954, a number of active radio frequency (RF) subsystems can be monitored. At step 956, a determination can be made as to whether the number of active RF subsystems have changed. If not the flow can return back to the monitoring step 954. If the number of active RF subsystems has changed, at step 958, the voltage thresholds can be changed for the EOL criteria. For example, referring back to FIG. 8, the threshold 804 can be increased or decreased a function of the EOL levels of the transceivers in single transmit operation (e.g. 806 and 808) and the multiple transmit EOL levels (e.g. 802). For instance, the threshold 804 can be adjusted to occur in the regions between the single transmit operations and the multiple transmit operations. Notably, the threshold 804 is adjusted in accordance with the EOL levels which may be inherently set in the transceivers.

Where applicable, the present embodiments of the invention can be realized in hardware, software or a combination of hardware and software. Any kind of computer system or other apparatus adapted for carrying out the methods described herein are suitable. A typical combination of hardware and software can be a mobile communications device with a computer program that, when being loaded and executed, can control the mobile communications device such that it carries out the methods described herein. Portions of the present method and system may also be embedded in a computer program product, which comprises all the features enabling the implementation of the methods described herein and which when loaded in a computer system, is able to carry out these methods.

While the preferred embodiments of the invention have been illustrated and described, it will be clear that the embodiments of the invention are not limited. Numerous modifications, changes, variations, substitutions and equivalents will occur to those skilled in the art without departing from the spirit and scope of the present embodiments of the invention as defined by the appended claims.

What is claimed is:

1. A method to handle early end of life (EOL) battery indications on a mobile device, comprising:
receiving an indication for an end of life (EOL) event;
determining an operating mode in view of the EOL event;
determining whether the mobile device is in muliple transmit communication;
evaluating a battery level of the mobile device; and
performing an action in response to the EOL event for mitigating an early shutdown of the mobile device based on the operating mode, the battery level, or the multiple transmit communication for allowing continued current drain on the battery and prolonging a use of the mobile device.

2. The method of claim 1, wherein an operating mode is at least one of an idle mode, an active mode, or an airplane mode.

3. The method of claim 1, wherein evaluating a battery level includes:
comparing a battery voltage to at least one threshold.

4. The method of claim 1, wherein performing an action includes:
masking a low battery alert corresponding to the EOL event.

5. The method of claim 1, wherein performing an action includes:
limiting at least one service associated with the operating mode.

6. The method of claim 1, wherein performing an action includes:
shutting down the mobile device.

7. The method of claim 1, wherein performing an action further comprises:
determining a service associated with the EOL event; and
limiting the service by changing at least one operating mode during the multiple transmit communication.

8. A method for handling low battery conditions on a mobile device, comprising:
monitoring the mobile device for an end of life (EOL) event;
determining an operating mode of at least one transceiver associated with the EOL event;
determining whether the EOL event is due to a multi-transceiver configuration;
evaluating a battery level of the mobile device; and
performing an action in response to the EOL event for mitigating an early shutdown of the mobile device based on the operating mode, the multi-transceiver configuration, or the battery level.

9. The method of claim 8, wherein performing an action includes,
if the device is operating in a multi-transceiver configuration:
measuring a battery capacity;
comparing the battery capacity to at least one threshold, and
changing an operating mode of the mobile device if the battery capacity exceeds at least one threshold in response to the EOL event.

10. The method of claim 9, wherein the changing an operating mode includes:
masking a low battery alert corresponding to the EOL event.

11. The method of claim 9, wherein the changing an operating mode includes:
limiting at least one service associated the operating mode.

12. The method of claim 9, wherein the changing an operating mode includes shutting down the mobile device.

13. The method of claim 9, wherein the measuring the battery capacity further comprises averaging multiple software readings of a voltage of the battery.

14. The method of claim 8, wherein a service is an active session or a network mobility update.

15. A mobile device to control early battery end of life events, comprising:
a battery for providing power to the mobile device;
at least one transceiver cooperatively coupled to the battery for providing communications; and
a processor coupled to the at least one transceiver and the battery for
monitoring an end of life (EOL) event due to multiple transmit activity of the at least one transceiver;
determining an operating mode of the at least one transceiver associated with the EOL event; and
masking the EOL event in view of the operating mode.

16. The mobile device of claim 15, further comprising:
a battery indicator for measuring a voltage of the battery,
wherein the processor compares the battery voltage to at least one threshold, and controls an operating mode of the mobile device if the battery voltage falls below at least one threshold.

17. The mobile device of claim 15, wherein the at least one transceiver generates an interrupt in response to current drain on the battery, wherein the interrupt is handled by the processor.

18. The mobile device of claim 15, wherein determining an operating mode includes determining whether the EOL event is due to activity of a multi-transceiver operation.

19. The mobile device of claim 18, wherein a first and second transceiver transmit concurrently over one of a CDMA, iDEN, WLAN, WiMax, WiDEN, or Bluetooth communication.

20. The mobile device of claim 18, further comprising:
a hardware detector cooperatively coupled to the processor to latch a state of the at least one transceiver, wherein the processor determines if at least two transceivers are concurrently active and causing the EOL event.
